(19) United States
(12) Patent Application Publication
(10) Pub. No.: US 2009/0264679 A1
(43) Pub. Date: Oct. 22, 2009

(54) Control of metal catalyst settling rates, settling densities and improved performance via use of flocculants

(76) Inventors: Daniel Ostgard, Kleinostheim (DE); Monika Berweiler, Maintal (DE); Barbara Bender, Rodenbach (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 11/721,648

(22) PCT Filed: Dec. 14, 2004
(86) PCT No.: PCT/EP2004/014212

§ 371 Date: May 15, 2009

(51) Int. Cl. B01J 25/00; C07C 5/03; B01J 27/20; C07C 209/30; C07C 209/32; C07C 29/132; B01D 21/01

(52) U.S. Cl. 564/494; 502/439; 502/301; 502/174; 585/275; 564/489; 568/861; 210/702

(57) ABSTRACT

A process for the adjustment of a catalyst's or a catalyst precursor's suspension and settling properties, whereby the catalyst is treated with flocculants.

### INTRODUCTION AND BACKGROUND

The present invention relates to the use of metal catalysts for the transformations of organic compounds, where the catalyst exhibits optimized settling rates and the desired settling density. The settling rate of the catalyst and its final settling density are very important factors involved in the use of these catalysts for a large number of transformation of organic compounds. Examples of these transformations include hydrogenations, hydrations, dehydrogenations, dehydrations, reductive aminations, reductive alkylations, isomerizations, oxidations, hydrogenolysis reactions and other commonly known reactions. Since many processes that involve metal catalysts use sedimentation as a method for the separation of the catalyst from the reaction mixture, the settling rate of the catalyst is critical to the overall reaction process time, in this case it is most desirable to have a fast settling rate. In some cases it may be better to have a slower settling rate. One such case involves the separation of the catalyst from the reaction mixture via filtration. In this case, it could be advantageous to have the catalyst remain suspended over the filter while a majority of the filtrate is pulled through the filter, especially if the catalyst settles down to form a very dense catalyst bed where it would then become difficult to pull the filtrate through.

The catalyst's settling density is also an important factor to be considered when choosing the appropriate catalyst for the process technique being employed. A loosely packed low-density catalyst bed is desired when the reslurrying of the catalyst back into suspension is a critical issue for the process. A loosely packed catalyst bed with a considerable amount of void spaces permits the reaction medium to permeate more freely throughout the catalyst bed, thereby allowing this liquid to lift the catalyst particles more readily into the reaction medium. This not only decreases the time for reslurrying, but is also cuts down on the maintenance of the stirring equipment, due to the lower workload on the stirrer's motor, and the amount of energy required to reach the desired suspension required for the optimal performance of the reaction. A loosely packed catalyst bed is also desired when the technology used for the separation of the catalyst from the reaction mixture requires that the reaction medium flows through the catalyst bed as smoothly as possible. An example of this would be the filtration of the reaction medium from the solid catalyst. Loosely packed metal catalyst beds are also more readily washed via the percolation or the flowing of the washing solution through the catalyst bed to be washed. Hence the ability to control the catalyst bed density during the preparation of the catalyst will lead to faster production times and better washed catalysts.

In some cases a more compact catalyst bed with a high packed density is desired. It is always necessary to remove as much water as possible when performing a water sensitive reaction. This is usually performed via the rinsing of the catalyst with a water miscible miscible organic solvent. This rinse is performed by allowing the catalyst to settle, decantation of the overstanding aqueous solution (usually performed by siphoning the overstanding solution away from the settled catalyst), the addition of a water miscible replacement solvent, the suspension is then stirred, allowed to settle and the overstanding solution is then once more decanted. The level of water in the catalyst is then stepwise decreased as the sequence of settling, decantation and washing is repeated and if a immiscible solvent is necessary, then this sequence needs to be repeated a few more times for the replacement of the water miscible solution with the desired one. In this sequence of events it is highly desirable to have a tightly packed catalyst bed that is as free as possible of any void spaces, so that the removal of the undesired solution is maximized for every cycle of sedimentation and decantation. Usually tight settling catalysts with high settling densities tend not to agglomerate during settling and this results in slower settling times. However, through the application of the invention of this patent it is possible to control the settling and density properties of the catalyst so that one could obtain a catalyst that agglomerates during settling for faster settling times while maintaining the advantages of a tightly packed catalyst bed with a high settling density.

It is also desirable to have a fast settling catalyst that forms a tightly compacted settled catalyst bed when embedding catalysts in materials that are liquid at temperatures somewhat above room temperature (preferably >50° C., but lower temperature melting materials are also useable in some cases) and solid at room temperature. This technology is typically used for the fatty amine embedding of Raney-type catalysts to be used for the hydrogenation of fatty nitriles to fatty amines. These embedded catalysts are made by initially allowing the catalyst to settle, the siphoning off of the catalyst's overstanding solution, the removal of most of the residual water via heating in a vacuum, the addition of the melted embedding agent (e.g., a suitable fatty amine), the homogenization of the embedding agent/catalyst mixture and the pastilation of the liquid mixture to droplet shaped solid embedded masses on a cooled conveyer. Having a rapidly settling catalyst that forms a tight bed will make this process faster and allow one to remove more water via siphoning and less by evacuation at higher temperatures. Since the removal of water by vacuum at higher temperature is slower and more expensive than by siphoning, the use of an appropriate flocculant will lead to a more commercially competitive catalyst embedding process.

The settling rate and the density of the settled metal catalyst bed is determined via the charge-to-particle size ratio, where a high charge-to-particle size ratio forces the particles to repel each other thereby allowing them to settle without the formation of agglomerates that tend to have a low concentration of voids. This leads to a tightly packed bed with a high density that can be used as mentioned above. A very low charge-to-particle size ratio allows the metal particles to coalesce into agglomerates that settle fast and contain a high concentration of voids thereby leads to a loosely packed catalyst bed with a low packing density. Hence one can control the settling of the metal catalyst particles by governing the charge-to-particle size ratio. This can be accomplished by changing the amount of the charge and/or the size of the catalyst particle. While changing the catalyst particle size can help, it is not without its drawbacks where too large of a metal catalyst particle leads to too low of an activity (due to the lower concentration of active metal surface area outside of the pore system) and too small of a catalyst particle can cause difficulties with the separation of the catalyst from the reaction medium and the increased likelihood of global mass transfer effects in reactions such as hydrogenation. Such mass transfer effects could lead to the faster deactivation of the catalyst and a serious drop in the desired reaction's yield. Nonetheless, the use of particle size along with the modifiers mentioned in this patent are a part of the invention described here.

One can also modify the charge-to-particle size ratio by changing the charge of the particles. This could be done by adding a charged agent to the catalyst for either the creation of a charged surface or the neutralization of a charged surface. Most modifiers of this type tend to block the active surface area and reduce the activity of the catalyst while creating template effects that may not give the desired reaction yields. The invention of this patent uses the surprising effect of the addition of flocculants together with the particle size effects of the metal catalyst in order to control both the settling rate and the settled density of the metal catalyst bed.

The design of the flocculant will also control the size and void volume of the catalyst agglomerates formed during sedimentation and present in the resulting settled catalyst bed. Flocculants will attract catalyst particles and bring them into their network of charged centers, thereby acting as a template for the agglomerate. The interaction of the particles with the flocculant depends on the charged species in this soluble polymer (e.g., acrylate anionic or quaternary amine cationic monomers in a polyacrylamide backbone) and the number of particles and their spacing in the agglomerate will depend on the type of ionicity of the flocculent, the type(s) of charged monomer(s) in the flocculant, the number of charged monomers per polymer strand, the molecular weight of the flocculant and the most likely number of flocculant strands involved in the building of each agglomerate. By controlling these factors, one could determine the overall settling and compaction properties of the catalyst as it forms a settled out catalyst bed. These properties will also determine the ionic atmosphere around the catalyst particles and the corresponding agglomerates leading to an additional advantage during the use of the flocculant treated catalyst via the optimization of the interaction between the reactant and the catalytic surface. This will be especially useful during reactions that are accelerated via an ionic interaction of the moiety to be reacted. One example would be the faster hydrogenation of polarized carbonyl compounds to their corresponding alcohols. These specially flocculant-built ionic atmospheres around the catalyst particles and agglomerates can also improve the effectiveness of ionic additives that improve the activity and selectivity of catalysts. An example of this would be the improved activity and selectivity of the hydrogenation of nitriles to primary amines by the addition of bases such as NaOH, LiOH, ammonia and others, where the effectiveness of these base additives are improved by the ionic atmosphere around the catalyst particle provided by the flocculent. Another example of optimizing the reactant-to-catalyst interaction via the use of flocculants would be the enantioselective hydrogenation of a prochiral unsaturated moiety where the flocculant impedes the adsorption of one face of the molecule over the other resulting in the enrichment of one enantiomeric product in the reaction mixture. In this respect, this technology can also be used with fixed bed catalysts where the interaction of the reactant and/or the additive to the catalyst can be optimized for the best results.

The preferred flocculants used in this invention are polyacrylamides. Polyacrylamides may either be nonionic, anionic or cationic and all of these varieties can be used in the current invention. The charge polyacrylamides are produced via the copolymerization of acrylamide with charged comonomers to produce the desired type and concentration of charges. Anionic polyacrylamides can be produced with acrylates (such as but not limited to, sodium acrylates) being used as the negatively charged monomers and cationic polyacrylamides can be produced with unsaturated quaternary amines that are readily polymerized. Many types of negatively and positively charged monomers are available for the production of the above mentioned charged polyacrylamides and the invention of this patent is not limited to those mentioned above. Theses flocculants are typically classified according to their charge and the density of this charge as determined by the concentration of the charged monomers present in the polyacrylamides. Flocculants are available worldwide and they are available under many different names. Although the flocculants used in this patent are from the Praestol® line of Degussa's bleach and water chemicals business unit, this patent also covers all the other flocculants regardless of their trade name. Flocculants other than polyacrylamides are also cover within the scope of this patent (e.g., polyacrylic acids as well as other flocculants that are not based on acrylamide or acrylic polymers).

These soluble flocculants may be added as powders, as a previously dissolved solution or as an emulsion to the catalyst treatment solution. The flocculants may also be dissolved as either powders, previously dissolved solutions, or emulsions into a catalyst suspension and one could adjust the settling properties of a larger suspension of catalyst via the addition of a certain amount of the treated catalyst to the larger catalyst suspension. One could also adjust the settling properties of a catalyst suspension by treating a suspension of another material and by adding the suspension of the other material to the catalyst suspension.

The type, the charge density and the amount of the desired flocculent can be determined via a series of tests where all the possible varieties of the above mentioned properties are tested on a series of metal catalyst samples of the same sample size under the same conditions of settling were the settling rate and the settling density are both determined. One type of experiment used in the optimization of the flocculant would be a modified jar-test where all the catalyst samples are stirred in the same sized jars under the same conditions upon addition of the flocculent followed by optionally a change in the stirring rate to allow for agglomeration maturity before the stirrer is turned off and the settling rates and settling densities are measured. This method can be improved by the use of a thinner jar that is calibrated and mixing can be achieved quicker via shaking this container with the treated catalyst. Graduate cylinders are ideal for such tests where the determination of the catalyst's sedimentation level after a certain time upon the cessation of mixing and the overall volume of this pre-weighed settled catalyst bed can be used to determine the settling rate and settling density of the treated catalyst respectively. Other types of experiments including modified stirrer, shaking, plunger mixing, vacuum filtration, filtration, sieving, capillary suction, plunger press and other methods known in the art were the settling speed, the settling density and/or the removal speed of the suspension's solution is measured to determine the desired flocculant and its amount.

Factors such as the initial water hardness, the suspension pH, the mixing dynamics and other critical parameters can be optimized via the series of experiments as described above.

After finding the appropriate flocculent, its amount and its conditions of addition, the treated metal catalyst is then tested to make sure that it maintains the desired level of activity. In some cases, the flocculant may improve the performance of the catalyst via an enhanced catalyst-to-reactant and/or -additive interaction (vide-supra). In such cases, this technology could also be used to improve fixed bed catalysts. It was also found that the flocculant can be used as an aid for the promotion of the catalyst with one or more elements from the periodic groups 1A, 2A, IIIB, IVB, VB, VIB, VIIB, VIII, IB, IIB, IIIA, IVA, VA and VIA. In these cases, the flocculant may be added to the catalyst before, during and/or after the addition of the promoting element precursor and still have the same desired effect. The use of flocculants for the promotion of catalysts with one or more elements of the periodic groups 1A, 2A, IIIB, IVB, VB, VIB, VIIB, VIII, IB, IIB, IIIA, IVA, VA and VIA may also be used for fixed bed catalysts.

The types of catalysts that can be improved by such a treatment include metal powder catalysts, catalytic metal blacks, metal boride catalysts, Raney-type metal catalysts, Ushibara type metal catalysts and other non-supported metal catalysts. The precursors of the above mentioned catalysts can also be treated with flocculants so that they can be better dispersed, promoted and interacted with the catalyst preparation medium. The metal powdered catalysts can be formed either by mechanical milling methods or chemical methods. The catalytic metal blacks are formed via the reduction of their metal salts in aqueous solution with hydrogen, formaldehyde, formic acid, sodium formate, hydrazine or other appropriate reducing agents. The catalytic metal borides are formed via the reduction of their metal salts in aqueous solution with sodium or potassium borohydride. The Ushibara catalysts are prepared via the precipitation of a metal salt (most commonly Ni) with zinc followed by a leaching treatment with either an acid or a base to give the active skeletal-type of Ushibara catalyst. The catalysts included in this invention contain one or more of the elements from the periodic groups 1A, 2A, IIIB, IVB, VB, VIB, VIIB, VIII, IB, IIB, IIIA, IVA, VA and VIA.

Activated metal catalysts are also known in the fields of chemistry and chemical engineering as Raney-type, sponge and/or skeletal catalysts. These powdered catalysts are prepared from an alloy of a catalytically active metal, also referred to herein as the catalyst metal, with a further alloying component that is soluble in alkalis. Mainly nickel, cobalt, copper, or iron are used as catalyst metals. The potential catalyst metals include those coming from the periodic groups VIII and IB. Aluminum is generally used as the alloying component which is soluble in alkalis, but other components may also be used, in particular zinc and silicon or mixtures of these either with or without aluminum.

These so-called Raney alloys are generally prepared by the ingot casting process. In that process a mixture of the catalyst metal and, for example, aluminum are first melted and casted into ingots. Typical alloy batches on a production scale amount to about ten to one hundred kg per ingot. According to DE 21 59 736 cooling times of up to two hours were obtained. This corresponds to an average rate of cooling of about 0.2 K/s. In contrast to this, rates of 102 to 106 K/s and higher are achieved in processes where rapid cooling is applied (for example an atomizing process). The rate of cooling is affected in particular by the particle size and the cooling medium (see Materials Science and Technology edited by R. W. Chan, P. Haasen, E. J. Kramer, Vol. 15, Processing of Metals and Alloys, 1991, VCH-Verlag Weinheim, pages 57 to 110). A process of this type is used in EP 0 437 788 B 1 in order to prepare a Raney alloy powder. In that process the molten alloy at a temperature of 5 to 500° C. above its melting point is atomized and cooled using water and/or a gas. The invention of this patent can be applied to the catalysts prepared from slowly, moderately and rapidly cooled alloys. The use of cooling mediums; including but not limited to water, air and inert gases (e.g., Ar, He, N2 and others); can also be used in fabricating the alloys that are activated with caustic solutions in order to generate the catalysts being modified for the desired settling and density characteristics.

To prepare a Raney-type catalyst, the Raney alloy is first finely milled if it has not been produced in the desired powder form during preparation. Then the aluminum is partly (and if need be, totally) removed by extraction with alkalis such as, for example, caustic soda solution (other bases such as KOH are also suitable) to activate the alloy powder. Following extraction of the aluminum the remaining catalytic power has a high specific surface area (BET), between 5 and 150 m²/g, and is rich in active hydrogen. The activated catalyst powder is pyrophoric and stored under water or organic solvents.

This invention also includes supported type powder catalysts where its settling and density properties are influenced by its charge-to-particle size ratio.

All of the catalysts mentioned above can also be promoted with one or more elements coming from the periodic groups 1A, 2A, IIIB, IVB, VB, VIB, VIIB, VIII, IB, IIB, IIIA, IVA, VA and VIA. Preferably the promoting elements come from the periodic groups IIIB, IVB, VB, VIB, VIIB, VIII, IB, IIB, IIIA, IVA and VA.

For the Raney type of catalysts, one or more of these promoting elements can be incorporated into the catalyst by either initially adding the element(s) to the precursor alloy before leaching or by adsorbing the element(s) either during or after the activation of the catalyst. Promotion with combinations of the above mentioned elements can also be accomplished by using a combination of techniques where one or more element(s) are added into the alloy and the other(s) or more of the same are/is added during or after leaching the alloy with caustic solutions.

This patent also includes the use of these flocculants for the improved promotion of the above mentioned catalysts with the above mentioned elements.

For additional details about these catalyst types and their promoted forms, please see R. L. Augustine, “Heterogeneous catalysis for the synthetic chemist”, Marcel Dekker, N.Y.

The addition of flocculants for the improved settling and density properties of the catalyst can be done during catalyst preparation, catalyst washing, catalyst use and catalyst recycling. In the case of Raney-type catalysts, the flocculent can be given to the alloy before activation, during the activation procedure and/or the washing procedure during the manufacture of this catalyst. Since the flocculant improves the suspension of the Raney-alloy in water, it is advantageous to add the flocculant to the alloy suspension before it is pumped into the activation reactor when the alloy is given to the activation medium as a slurry. As mentioned above, the flocculant can be used to enhance the addition of one or more promoters to the catalyst or its precursor. The flocculant can also be added to the finished catalyst. The flocculent can be quickly added to the stirred suspension of the catalyst in its drum prior to use. The flocculent can also be given to reaction mixture at the beginning of the reaction, during the reaction or at the end of the reaction in order to ensure the proper settling behavior and settling density properties of the catalyst. Another version of this invention includes the addition of the flocculant to the reaction mixture after the reaction is finished so that the separation of the catalyst from this reaction medium is carried out as desired. If the catalyst has a problem with catalyst fines, the use of flocculants will help in the formation of agglomerates that contain these fines so that they too will settle faster. In some cases, it is advisable to remove as many fines as possible from the catalyst and this may be done quickly by initially allowing the catalyst to settle, siphoning the overstanding solution above the settled catalyst into a sedimentation tank and the addition flocculants to this suspension with the fines. The flocculent will assist in the sedimentation of the fines so that they can be removed from the suspension. The flocculant can also be given to the catalyst during its recycling process. The addition of the optimized flocculant to the catalyst can be performed during any combination or all of the above mentioned time points that occur during the production, use and recycling of the catalyst.

### SUMMARY OF THE INVENTION

The above and other objects of this invention are carried out by the addition of one or more flocculants to metal catalysts and their precursors for the optimization of their settling and density properties. The types of catalysts that can be improved by such a treatment include metal powder catalysts, catalytic metal blacks, metal boride catalysts, Raney-type metal catalysts, Ushibara type metal catalysts and other non-supported metal catalysts. The settling and density properties of supported catalysts and their precursors can also be improved with the invention of this patent, especially when these propertied depend on the charge-to-particle size properties of the catalyst. The catalysts included in this invention contain one or more of the elements from the periodic groups 1A, 2A, IIIB, IVB, VB, VIB, VIIB, VIII, IB, IIB, IIIA, IVA, VA and VIA. This invention also includes catalysts that were doped with the above mentioned elements via the addition of flocculants. The application of the flocculants can be performed during the preparation, washing, use and recycling of these catalysts as well as with their precursors. In the case of Raney-type catalysts, the optimized flocculent can be added before alloy activation, during alloy activation to the catalyst, during catalyst washing, during catalyst promotion, during catalyst use, after catalyst use during filtration, during catalyst recycling, during some of these timeframes and/or during all of these timeframes. This invention includes all types of flocculants. One class of preferred flocculants is based on neutral, anionic and cationic polyacrylamides. The flocculants can be used as granules, emulsions, water solutions, oil free dispersions or any other commonly used form. One could add the flocculent to the catalyst suspension as a powder, a previously dissolved solution, an emulsion, as part of a treated catalyst suspension that will be added to the catalyst suspension that one wants to modify and/or as part of treated suspension of another material that can be added to the catalyst suspension. Although the Praestol® products of Degussa are used in the examples below, the invention of this patent includes all the flocculants from the other vendors as well. These flocculants can also be used for the improved promotion of the above-mentioned catalysts with one or more elements from the periodic groups 1A, 2A, IIIB, IVB, VB, VIB, VIIB, VIII, IB, IIB, IIIA, IVA, VA and VIA.

The flocculants mentioned here are used to optimize the settling density and the settling speed of the catalyst. It is very advantageous to have a rapidly settling catalyst that forms a low volume bed when the water solution above the catalyst needs to be exchanged for another solvent. This is especially advantageous when the catalyst needs to be embedded in materials such as fatty amines. The flocculants used here can also be used to design the size, type and amount of void space in the catalyst particle agglomerates. The properties of the flocculants can also control the interaction of the catalyst with the reactant and the reaction additives so that the reaction proceeds more selectively at a higher rate. It has also been found that flocculants can lead to the preferential hydrogenation of one face of a prochiral unsaturated molecule over the other resulting in a higher enantioselectivity of the reaction. Although fixed bed catalysts do not necessarily need improved settling properties, this invention also includes the use of flocculants with fixed bed catalysts for their improved doping with the elements mentioned above, as well as, for the improve interaction of the fixed bed catalyst with the reactant and reaction additives for the enhanced performance of the fixed bed catalyst (e.g., improved activity, selectivity, enantioselectivity and others) during the desired reaction.

### EXAMPLE 1

The treatment of a Raney-type Ni catalyst having an average particle size of ˜28 μm with flocculants where the original settling density of the moist catalyst cake was 1.14 g/ml.

Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 1.

TABLE 1
The settling properties of the flocculant treated
Raney-type Ni catalysts of example 1 having an average
particle size of ~28 μm where the original settling density
of the moist catalyst cake was 1.14 g/ml.

| | | ml | | Relative | Settled | Overstanding |
|---|---|---|---|---|---|---|
| Sample | Flocculant | of 0.05 wt % | | settling | volume, | Solution |
| number | Type* | flocculant | Agglomerating | rate | ml | After 15 min |
| 1a | — | 0.0 | yes | fast | 36 | Clear |
| 1b | 852 BC | 10 | yes | fast | 26.5 | Clear |
| | cationic | | | | | |
| 1c | 806 BC | 10 | yes | fast | 29 | Clear |
| | cationic | | | | | |
| 1d | 2515 | 2 | yes | Very fast | 30 | clear |
| | anionic | | | | | |
| 1e | 2515 | 2.5 | yes | Very fast | 29.5 | clear |
| | anionic | | | | | |

*Praestol ® flocculants from Degussa.

### EXAMPLE 2

The treatment of a Raney-type Ni catalyst having an average particle size of ˜28 μm with flocculants where the original settling density of the moist catalyst cake was 1.90 g/ml.

The catalyst used in this example was prepared with very hard water that contained a considerable amount of minerals and cations. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculant solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 2.

TABLE 2
The settling properties of the flocculant treated
Raney-type Ni catalysts of example 2 having an average
particle size of ~28 μm where the original settling density
of the moist catalyst cake was 1.90 g/ml.

| | | ml | | Relative | Settled | Overstanding |
|---|---|---|---|---|---|---|
| Sample | Flocculant | of 0.05 wt % | | settling | volume, | Solution |
| number | Type* | flocculant | Agglomerating | rate | ml | After 15 min |
| 2a | — | 0.0 | no | Very slow | 21 | Very murky |
| 2b | 806 BC | 10 | yes | Very fast | 27 | Clear |
| | cationic | | | | | |
| 2c | 2515 | 10 | yes | Very fast | 31 | clear |
| | anionic | | | | | |
| 2d | 2515 | 2 | yes | fast | 28 | clear |
| | anionic | | | | | |

*Praestol ® flocculants from Degussa.

### EXAMPLE 3

The treatment of a Raney-type Ni catalyst having an average particle size of ˜53 μm with flocculants where the original settling density of the moist catalyst cake was 1.67 g/ml.

Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 3.

TABLE 3
The settling properties of the flocculant treated
Raney-type Ni catalysts of example 3 having an average
particle size of ~53 μm where the original settling density
of the moist catalyst cake was 1.67 g/ml.

| | | ml of | | Relative | | Overstanding |
|---|---|---|---|---|---|---|
| Sample | Flocculant | 0.05 wt % | | settling | Settled | Solution |
| number | Type* | flocculant | Agglomerating | rate | volume, ml | After 15 min |
| 3a | — | 0.0 | no | slow | 24 | turbid |
| 3b | 2540 | 2 | yes | fast | 29.5 | clear |
| | anionic | | | | | |
| 3c | 2515 | 5 | yes | fast | 28 | clear |
| | anionic | | | | | |

*Praestol ® flocculants from Degussa.

### EXAMPLE 4

The treatment of a Raney-type Cu catalyst having an average particle size of ˜43 μm with flocculants where the original settling density of the moist catalyst cake was 1.43 g/ml.

Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 4.

TABLE 4
The settling properties of the flocculant treated
Raney-type Cu catalysts of example 4 having an average
particle size of ~43 μm where the original settling density
of the moist catalyst cake was 1.43 g/ml.

| | | ml | | Relative | Settled | Overstanding |
|---|---|---|---|---|---|---|
| Sample | Flocculant | of 0.05 wt % | | settling | volume, | Solution |
| number | Type* | flocculant | Agglomerating | rate | ml | After 15 min |
| 4a | — | 0.0 | no | normal | 28 | clear |
| 4b | 806 BC | 10 | yes | Very fast | 31 | clear |
| | cationic | | | | | |
| 4c | 2540 | 2 | yes | fast | 35 | clear |
| | anionic | | | | | |

*Praestol ® flocculants from Degussa.

### EXAMPLE 5

The treatment of a Raney-type Co catalyst having an average particle size of ˜38 μm with flocculants where the original settling density of the moist catalyst cake was 1.60 g/ml.

Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 5.

TABLE 5
The settling properties of the flocculant treated
Raney-type Co catalysts of example 5 having an average
particle size of ~38 μm where the original settling density
of the moist catalyst cake was 1.60 g/ml.

| | | ml of | | Relative | Settled | Overstanding |
|---|---|---|---|---|---|---|
| Sample | Flocculant | 0.05 wt % | | settling | volume, | Solution |
| number | Type* | flocculant | Agglomerating | rate | ml | After 15 min |
| 5a | — | 0.0 | no | slow | 24 | turbid |
| 5b | 2515 anionic | 10 | yes | Very fast | 23.5 | clear |
| 5c | 806 BC | 10 | yes | Very fast | 21 | clear |
| | cationic | | | | | |
| 5d | 2515 anionic | 2 | yes | Very fast | 21 | clear |
| 5e | 806 BC | 2 | yes | fast | 20.5 | clear |
| | cationic | | | | | |
| 5f | 2540 anionic | 10 | yes | fast | 30 | clear |

*Praestol ® flocculants from Degussa.

### EXAMPLE 6

The treatment of an Fe doped Raney-type Ni catalyst having an average particle size of ˜53 μm with flocculants where the original settling density of the moist catalyst cake was 1.54 g/ml and the preactivated alloy contained the doping element.

This treatment was carried out with a Fe doped Raney-type Ni catalyst containing about ˜11% Fe, where the Fe was already present in the preactivated alloy. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 6.

TABLE 6
The settling properties of the flocculant treated
Fe doped Raney-type Ni catalysts of example 6 having an
average particle size of ~53 μm where the original settling
density of the moist catalyst cake was 1.54 g/ml.

| | | ml | | Relative | Settled | Overstanding |
|---|---|---|---|---|---|---|
| Sample | Flocculant | of 0.05 wt % | | settling | volume, | Solution |
| number | Type* | flocculant | Agglomerating | rate | ml | After 15 min |
| 6a | — | 0.0 | no | fast | 26 | murky |
| 6b | 2515 anionic | 10 | yes | fast | 30 | clear |
| 6c | 2540 anionic | 10 | yes | fast | 29 | clear |
| 6d | 2515 anionic | 2 | yes | fast | 26 | clear |
| 6e | 2540 anionic | 2 | yes | fast | 24 | clear |

*Praestol ® flocculants from Degussa.

### EXAMPLE 7

The treatment of an Fe and Cr doped Raney-type Ni catalyst having an average particle size of ˜33 μm with flocculants where the original settling density of the moist catalyst cake was 1.60 g/ml and the preactivated alloy contained the doping elements.

This treatment was carried out with a Fe and Cr doped Raney-type Ni catalyst, where the Cr and Fe were already present in the preactivated alloy at a Cr-to-Fe weight ratio of 5-to-1. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculant solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 7.

TABLE 7
The settling properties of the flocculant treated
Cr and Fe doped Raney-type Ni catalysts of example 7 having
an average particle size of ~33 μm where the original
settling density of the moist catalyst cake was 1.60 g/ml.

| | | | | Relative | | Overstanding |
|---|---|---|---|---|---|---|
| Sample | Flocculant | ml of 0.05 wt % | | settling | Settled | Solution After |
| number | Type* | flocculant | Agglomerating | rate | volume, ml | 15 min |
| 7a | — | 0.0 | no | Very slow | 25 | Very murky |
| 7b | 806 BC | 10 | yes | fast | 34 | clear |
| | cationic | | | | | |
| 7c | 852 BC | 10 | yes | very fast | 24 | clear |
| | cationic | | | | | |
| 7d | 806 BC | 2 | yes | very fast | 23.5 | clear |
| | cationic | | | | | |
| 7e | 852 BC | 2 | yes | very fast | 21.5 | clear |
| | cationic | | | | | |

*Praestol ® flocculants from Degussa.

### EXAMPLE 8

The treatment of an Fe and Cr doped Raney-type Ni catalyst having an average particle size of ˜33 μm with flocculants where the original settling density of the moist catalyst cake was 1.74 g/ml, the preactivated alloy contained the doping elements and the catalyst was pretreated with flocculent during the washing step of its preparation.

This treatment was carried out with a Fe and Cr doped Raney-type Ni catalyst, where the Cr and Fe were already present in the preactivated alloy at a Cr-to-Fe weight ratio of 5-to-1. The only difference between the catalyst of this example and that of example 7 is that this catalyst was washed with the flocculant Praestol® 2515 during the final washing step of its preparation. During the final washing step, 100 grams of this catalyst were treated with 3 ml of a 0.05 wt % Praestol® 2515 solution. Forty grams of this moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 8.

TABLE 8
The settling properties of the flocculant treated
Cr and Fe doped Raney-type Ni catalysts of example 8 having
an average particle size of ~33 μm where the original
settling density of the moist catalyst cake was 1.74 g/ml
and the catalyst was already treated with the flocculant
Praestol ® 2515 during its preparation.

| | | | | Relative | | Overstanding |
|---|---|---|---|---|---|---|
| Sample | Flocculant | ml of 0.05 wt % | | settling | Settled | Solution |
| number | Type* | flocculant | Agglomerating | rate | volume, ml | After 15 min |
| 8a | — | 0.0 | no | slow | 23 | murky |
| 8b | 806 BC | 10 | yes | fast | 36 | clear |
| | cationic | | | | | |
| 8c | 852 BC | 10 | yes | fast | 30 | clear |
| | cationic | | | | | |
| 8d | 806 BC | 2 | yes | very fast | 31 | clear |
| | cationic | | | | | |
| 8e | 852 BC | 2 | yes | very fast | 28 | clear |
| | cationic | | | | | |

*Praestol ® flocculants from Degussa.

### EXAMPLE 9

The treatment of a Fe and Cr doped Raney-type Ni catalyst having an average particle size of ˜33 μm with flocculants where the original settling density of the moist catalyst cake was 1.45 g/ml and the preactivated alloy contained the doping elements.

This treatment was carried out with an Fe and Cr doped Raney-type Ni catalyst, where the Cr and Fe were already present in the preactivated alloy at a Cr-to-Fe weight ratio of ˜1-to-1. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculant solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 9.

TABLE 9
The settling properties of the flocculant treated
Cr and Fe doped Raney-type Ni catalysts of example 9 having
an average particle size of ~33 μm where the original
settling density of the moist catalyst cake was 1.45 g/ml.

| | | | | Relative | | Overstanding |
|---|---|---|---|---|---|---|
| Sample | Flocculant | ml of 0.05 wt % | | settling | Settled | Solution |
| number | Type* | flocculant | Agglomerating | rate | volume, ml | After 15 min |
| 9a | — | 0.0 | yes | fast | 27.5 | clear |
| 9b | 2515 anionic | 5 | yes | fast | 31.5 | clear |
| 9c | 806 BC | 2 | yes | fast | 30 | clear |
| | cationic | | | | | |
| 9d | 806 BC | 5 | yes | fast | 34 | clear |
| | cationic | | | | | |
| 9e | 806 BC | 10 | yes | fast | 34 | clear |
| | cationic | | | | | |
| 9e | 806 BC | 15 | yes | fast | 35 | clear |
| | cationic | | | | | |

*Praestol ® flocculants from Degussa.

### EXAMPLE 10

The treatment of a Mo doped Raney-type Ni catalyst having an average particle size of ˜33 μm with flocculants where the original settling density of the moist catalyst cake was 1.13 g/ml and the preactivated alloy contained Mo. This treatment was carried out with a Mo doped Raney-type Ni catalyst, where the Mo was already present in the preactivated alloy. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculant solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 10.

TABLE 10
The settling properties of the flocculant treated
Mo doped Raney-type Ni catalysts of example 10 having an
average particle size of ~33 μm where the original settling
density of the moist catalyst cake was 1.13 g/ml and the
preactivated alloy contained Mo.

| | | | | Relative | | Overstanding |
|---|---|---|---|---|---|---|
| Sample | Flocculant | ml of 0.05 wt % | | settling | Settled | Solution |
| number | Type* | flocculant | Agglomerating | rate | volume, ml | After 15 min |
| 10a | — | 0.0 | yes | Fast | 35.5 | clear |
| 10b | 806 BC | 2 | yes | Fast | 28 | clear |
| | cationic | | | | | |
| 10c | 806 BC | 5 | yes | Very fast | 24 | clear |
| | cationic | | | | | |

*Praestol ® flocculants from Degussa.

### EXAMPLE 11

The treatment of a Mo doped Raney-type Ni catalyst having an average particle size of ˜53 μm with flocculants where the original settling density of the moist catalyst cake was 2.11 g/ml and the catalyst was doped after activation.

This treatment was carried out with a Mo doped Raney-type Ni catalyst, where the catalyst was first activated and then doped with a sodium molybdate salt to the concentration of 2% Mo. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 11.

TABLE 11
The settling properties of the flocculant treated
Mo doped Raney-type Ni catalysts of example 11 having an
average particle size of ~53 μm where the original settling
density of the moist catalyst cake was 2.11 g/ml and the
catalyst was doped after activation.

| | | | | Relative | Settled | Overstanding |
|---|---|---|---|---|---|---|
| Sample | Flocculant | ml of 0.05 wt % | | settling | volume, | Solution After |
| number | Type* | flocculant | Agglomerating | rate | ml | 15 min |
| 11a | — | 0.0 | no | very slow | 19 | Very murky |
| 11b | 806 BC | 2 | yes | fast | 27 | Clear |
| | cationic | | | | | |
| 11c | 806 BC | 4 | yes | fast | 28 | Clear |
| | cationic | | | | | |
| 11d | 806 BC | 10 | yes | fast | 32 | Clear |
| | cationic | | | | | |
| 11e | 2515 anionic | 5 | yes | very fast | 23 | Clear |

*Praestol ® flocculants from Degussa.

### EXAMPLE 12

The treatment of a Mo doped Raney-type Ni catalyst having an average particle size of ˜53 μm with flocculants where the original settling density of the moist catalyst cake was 2.05 g/ml and the catalyst was doped after activation.

This treatment was carried out with a Mo doped Raney-type Ni catalyst, where the catalyst was first activated and then doped with an ammonium molybdate salt to the concentration of 2.5% Mo. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 12.

TABLE 12
The settling properties of the flocculant treated
Mo doped Raney-type Ni catalysts of example 12 having an
average particle size of ~53 μm where the original settling
density of the moist catalyst cake was 2.05 g/ml and the
catalyst was doped after activation.

| | | | | Relative | Settled | Overstanding |
|---|---|---|---|---|---|---|
| Sample | Flocculant | ml of 0.05 wt % | | settling | volume, | Solution After |
| number | Type* | flocculant | Agglomerating | rate | ml | 15 min |
| 12a | — | 0.0 | no | very slow | 19.5 | very murky |
| 12b | 2515 anionic | 10 | yes | very fast | 24 | clear |
| 12c | 2515 anionic | 10 | yes | very fast | 24 | clear |
| 12d | 806 BC | 10 | yes | very fast | 24 | clear |
| | cationic | | | | | |
| 12e | 852 BC | 10 | yes | very fast | 24.5 | clear |
| | cationic | | | | | |
| 12f | 2515 anionic | 2 | yes | fast | 24 | clear |
| 12g | 2540 anionic | 2 | yes | fast | 24.5 | clear |
| 12h | 806 BC | 2 | yes | fast | 23.5 | clear |
| | cationic | | | | | |
| 12i | 852 BC | 2 | yes | fast | 24 | clear |
| | cationic | | | | | |

*Praestol ® flocculants from Degussa.

### EXAMPLE 13

The use of flocculants for the improved Mo doping of an activated Raney-type Ni catalyst with MoO3, where the average particle size is ˜53 μm.

Forty grams of the undoped moist catalyst cake (23.5 grams on a dry basis) were weighed out and placed into a graduate cylinder. The graduate cylinder was filled to a volume of 80 ml with distilled water, the desired amount of a 0.05 wt. % flocculent solution and MoO3 were then added and the total volume was made up to 100 ml with distilled water. The amount of MoO3 added to the catalyst was enough to give the catalyst a 1 wt. % Mo content. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear and if it contained any dissolved Mo after 15 minutes of settling. This procedure was repeated for every type of flocculant addition that was tested and a control sample was also prepared where no flocculant was given to the catalyst. The type of flocculant, its amount and the settling properties of the corresponding catalysts of this type are listed in Table 13.

TABLE 13
The properties of the Mo doped Raney-type Ni
catalysts of example 13 having an average particle size of
~53 μm, where the Mo doping procedure was assisted by the
use of flocculants.

| | | | | Relative | | Overstanding |
|---|---|---|---|---|---|---|
| Sample | Flocculant | Ml of 0.05 wt % | | settling | Settled | Solution After |
| number | Type* | flocculant | Agglomerating | rate | volume, ml | 15 min |
| 13a | none | 0.0 | yes | very fast | 27 | very murky with |
| | | | | | | >100 ppm Mo |
| 13b | 2515 | 10 | yes | very fast | 28 | Clear with 0 ppm |
| | anionic | | | | | Mo |
| 13c | 2515 | 10 | yes | very fast | 28 | Clear with 0 ppm |
| | anionic | | | | | Mo |

*Praestol ® flocculants from Degussa.

### EXAMPLE 14

The use of flocculants for the improved Mo doping of an activated Raney-type Ni catalyst with MoO3, where the average particle size is ˜53 μm.

Eight hundred and fifty grams of a moist Raney-type Ni catalyst (500 grams catalyst on a dry basis) were mixed with 13.5 grams of MoO3, 220 ml of a 0.05 wt % Praestol® 806 BC flocculent solution and enough water to bring the total volume up to 800 ml. This mixture was then stirred for one hour, after which it was found that the overstanding solution above the settled catalyst contain 0 ppm Mo. Forty grams of the moist catalyst cake (23.5 grams on a dry basis) were weighed out and placed into a graduate cylinder. The graduate cylinder was filled to a volume of 100 ml with distilled water, a stopper was placed into the top of the graduate cylinder, it was shaken vigorously for 1 minute and the settling properties of the catalyst were then noted and measured. The catalyst was an agglomerating type that settled fast and the final level in the graduate cylinder was 32 ml giving a settled density of 1.25 g/ml for the catalyst cake. After 15 minutes of settling, the overstanding solution above the catalyst was clear and it did not contain Mo. This catalyst is referred to as sample 14 in this patent.

### EXAMPLE 15

The use of flocculants for the improved Mo doping of an activated Raney-type Ni catalyst with MoO3, where the average particle size is ˜53 μm.

Eight hundred and fifty grams of a Raney-type Ni catalyst (500 grams catalyst on a dry basis) were stirred in enough water to bring the total volume of this suspension up to 800 ml. In the meantime, 4 ml of a 50% NaOH solution were added to 80 ml of water and 13.5 grams of MoO3 were dissolved into this solution. After 5 minutes of stirring the catalyst suspension, the above mentioned Mo solution was added uniformly over 10 minutes to the catalyst followed by the addition of 110 ml of a 0.05 wt % Praestol® 806 BC flocculent solution and this was then stirred for an additional 30 minutes. An additional 110 ml of the 0.05 wt % Praestol® 806 BC flocculant solution was added to the catalyst suspension and this slurry was stirred for an additional 5 hours, after which it was found that the overstanding solution above the settled catalyst contain 0 ppm Mo. Forty grams of the moist catalyst cake (23.5 grams on a dry basis) were weighed out and placed into a graduate cylinder. The graduate cylinder was filled to a volume of 100 ml with distilled water, a stopper was placed into the top of the graduate cylinder, it was shaken vigorously for 1 minute and the settling properties of the catalyst were then noted and measured. The catalyst was an agglomerating type that settled fast and the final level in the graduate cylinder was 30 ml giving a settled density of 1.33 g/ml for the catalyst cake. After 15 minutes of settling, the overstanding solution above the catalyst was clear and it did not contain Mo. This catalyst is referred to as sample 15 in this patent.

### EXAMPLE 16

The use of flocculants for the improved suspension and settling properties of a formaldehyde modified Raney-type Ni catalyst, where the average particle size is ˜53 μm.

Eight hundred and fifty grams of a Raney-type Ni catalyst (500 grams catalyst on a dry basis) with an average particle size of ˜53 μm were mixed with 1 liter of water and stirred to form a homogeneous suspension. In the meantime, 105 ml of an aqueous technical grade 37% formaldehyde solution were mixed together with 225 ml of an aqueous 5% NaOH solution. This formaldehyde/NaOH solution was then added uniformly over 20 minutes to the catalyst slurry and this was followed by an additional hour of stirring. The catalyst was then allowed to settle and the overstanding solution was found to have 0 ppm Ni and 0 ppm formaldehyde. This catalyst was then washed twice with 1 liter of distilled water each time. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 14.

TABLE 14
The settling properties of the formaldehyde
treated Raney-type Ni catalysts of example 16 having an
average particle size of ~53 μm where the original settling
density of the moist catalyst cake was 1.90 g/ml.

| | | | | | Settled | Overstanding |
|---|---|---|---|---|---|---|
| Sample | Flocculant | ml of 0.05 wt % | | Relative | volume, | Solution After |
| number | Type* | flocculant | Agglomerating | settling rate | ml | 15 min |
| 16a | — | 0.0 | no | very slow | 21 | murky |
| 16b | 2515 | 10 | yes | very fast | 30 | clear |
| | anionic | | | | | |
| 16c | 806 BC | 10 | yes | very fast | 28 | clear |
| | cationic | | | | | |

*Praestol ® flocculants from Degussa.

### EXAMPLE 17

The use of flocculants for the improved suspension and settling properties of a formaldehyde modified Raney-type Ni catalyst, where the average particle size is ˜53 μm.

Eight hundred and fifty grams of a Raney-type Ni catalyst (500 grams catalyst on a dry basis) with an average particle size of ˜53 μm were stirred as part of an 800 ml slurry to form a homogeneous suspension. Exactly 112,5 ml of an aqueous technical grade 37% formaldehyde solution were then added uniformly over 5 minutes to the catalyst slurry and this was followed by an additional hour of stirring. The catalyst was then allowed to settle and samples were taken for flocculant treatments. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 15.

TABLE 15
The settling properties of the formaldehyde
treated Raney-type Ni catalysts of example 17 having an
average particle size of ~53 μm where the original settling
density of the moist catalyst cake was 2.11 g/ml.

| | | | | Relative | | Overstanding |
|---|---|---|---|---|---|---|
| Sample | Flocculant | ml of 0.05 wt % | | settling | Settled | Solution |
| number | Type* | flocculant | Agglomerating | rate | volume, ml | After 15 min |
| 17a | — | 0.0 | no | slow | 19 | murky |
| 17b | 2515 anionic | 10 | yes | very fast | 23 | clear |
| 17c | 806 BC | 10 | yes | very fast | 23 | clear |
| | cationic | | | | | |
| 17d | 2515 anionic | 2 | yes | Fast | 26 | clear |
| 17e | 806 BC | 2 | yes | Very fast | 23 | clear |
| | cationic | | | | | |

*Praestol ® flocculants from Degussa.

### EXAMPLE 18

The use of flocculants for the improved suspension and settling properties of a formaldehyde modified Raney-type Ni catalyst, where the average particle size is ˜53 μm.

Eight hundred and fifty grams of a Raney-type Ni catalyst (500 grams catalyst on a dry basis) with an average particle size of ˜53 μm were stirred as part of an 800 ml slurry to form a homogeneous suspension. Exactly 112,5 ml of an aqueous technical grade 37% formaldehyde solution were then added uniformly over 5 minutes to the catalyst slurry and this was followed by an additional hour of stirring. The catalyst was then allowed to settle and samples were taken for flocculant treatments. Forty grams of the moist catalyst cake (23.5 grams on a dry basis) were weighed out and placed into a graduate cylinder. The graduate cylinder was filled to a volume of 80 ml with distilled water, the desired amount of a 0.05 wt. % flocculent solution and 2 ml of a 10 wt. % aqueous NaOH solution were then added and the total volume was then made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 16.

TABLE 16
The settling properties of the formaldehyde
treated Raney-type Ni catalysts of example 18 having an
average particle size of ~53 μm.

| | | | | | | Overstanding |
|---|---|---|---|---|---|---|
| Sample | Flocculant | ml of 0.05 wt % | | Relative | Settled | Solution |
| number | Type* | flocculant | Agglomerating | settling rate | volume, ml | After 15 min |
| 18a | — | 0.0 | no | slow | 20 | clear |
| 18b | 2515 anionic | 10 | yes | fast | 24 | clear |
| 18c | 806 BC | 10 | yes | very fast | 23 | clear |
| | cationic | | | | | |
| 18d | 2515 anionic | 2 | yes | fast | 23 | clear |
| 18e | 806 BC | 2 | yes | fast | 22.5 | clear |
| | cationic | | | | | |

*Praestol ® flocculants from Degussa.

### EXAMPLE 19

The use of flocculants for the improved suspension and settling properties of a spent Raney-type Ni catalyst that was promoted with Mo, where the average particle size is ˜28 μm.

An activated Ni catalyst that was doped with 1.2% Mo was recycled more than 50 times in a batch sugar hydrolysate hydrogenation process. Forty grams of this moist spent catalyst cake (23.5 grams on a dry basis) were weighed out and placed into a graduate cylinder. The graduate cylinder was filled to a volume of 80 ml with distilled water, the desired amount of a 0.05 wt. % flocculent solution was then added and the total volume was then made up to 100 ml with distilled water. A stopper was then placed into the top of the graduate cylinder, it was shaken vigorously for 1 minute and the settling properties of the catalyst were then noted and measured. It was noted if the catalyst settled either with or without the formation of agglomerates, the relative settling rate was observed and the final settled volume of the catalyst bed was written down. It was also noted if the overstanding solution of the suspension was murky or clear after 15 minutes of settling. This procedure was repeated for every type of flocculant addition that was tested and a control sample was also prepared where no flocculant was given to the catalyst. The type of flocculent, its amount and the settling properties of the corresponding catalysts of this type are listed in Table 17.

TABLE 17
The settling properties of spent Mo-doped Raney-
type Ni catalysts of example 19 having an average particle
size of ~28 μm.

| | | | | | | Overstanding |
|---|---|---|---|---|---|---|
| Sample | Flocculant | ml of 0.05 wt % | | Relative | Settled | Solution |
| number | Type* | flocculant | Agglomerating | settling rate | volume, ml | After 15 min |
| 19a | — | 0.0 | no | very slow | 18 | very murky |
| 19b | 2515 anionic | 10 | yes | fast | 29 | clear |
| 19c | 806 BC | 10 | yes | very fast | 29 | clear |
| | cationic | | | | | |
| 19d | 2515 anionic | 2 | yes | fast | 30 | clear |
| 19e | 806 BC | 2 | yes | fast | 28 | clear |
| | cationic | | | | | |

*Praestol ® flocculants from Degussa.

### EXAMPLE 20

The use of flocculants for the improved settling properties of a 5 wt % suspension of a spent Mo promoted Raney-type Ni catalyst in a 50 wt % aqueous sorbitol solution, where the average particle size of the catalyst is ˜28 μm.

An activated Ni catalyst that was doped with 1.2% Mo was recycled more than 50 times in a batch sugar hydrolysate hydrogenation process. This catalyst was used to prepare two 5wt % suspensions of this catalyst in 50 wt % aqueous sorbitol solutions. Each suspension was prepared by adding forty grams of this moist catalyst cake (23.5 grams on a dry basis) to 430 grams of a 50 wt % sorbitol solution. One suspension did not contain any additives and was used as a comparison, while the other was treated with 10 ml of a 0.05 wt % 806 BC cationic Praestol® solution. Both catalyst suspensions were stirred at room temperature to form homogeneous slurries and the stirring was stopped for both at the same time. The catalyst suspension with the 806 BC cationic Praestol® settled fast and produced a clear overstanding solution, however the catalyst slurry without the 806 BC cationic Praestol® settled slowly and did not produce a clear overstanding solution. These two suspensions were then heated to 60° C., stirred to homogeneity and the stirring of both of them were stopped at the same time. At this elevated temperature the catalyst suspension with 806 BC cationic Praestol® settled faster and produced a clearer overstanding solution in comparison to the suspension without the flocculent.

### EXAMPLE 21

The use of flocculants for the improved settling properties of a 5 wt % suspension of a spent Mo promoted Raney-type Ni catalyst in a 50 wt % aqueous glucose solution, where the average particle size of the catalyst is ˜28 μm.

An activated Ni catalyst that was doped with 1.2% Mo was recycled more than 50 times in a batch sugar hydrolysate hydrogenation process. This catalyst was used to prepare two 5wt % suspensions of this catalyst in 50 wt % aqueous glucose solutions. Each suspension was prepared by adding forty grams of this moist catalyst cake (23.5 grams on a dry basis) to 430 grams of a 50 wt % glucose solution. One suspension did not contain any additives and was used as a comparison, while the other was treated with 5 ml of a 0.10 wt % 806 BC cationic Praestol® solution. Both catalyst suspensions were stirred at room temperature to form homogeneous slurries and the stirring was stopped for both at the same time. The catalyst suspension with the 806 BC cationic Praestol® settled fast and produced a clear overstanding solution in 30 minutes, however the catalyst slurry without the 806 BC cationic Praestol® settled slowly and did not produce a clear overstanding solution. These two suspensions were then heated to 60° C., stirred to homogeneity and the stirring of both of them were stopped at the same time. At this elevated temperature the catalyst suspension with 806 BC cationic Praestol® settled faster and produced a clearer overstanding solution in comparison to the suspension without the flocculant.

### EXAMPLE 22

The use of flocculants for the improved settling properties of a 5 wt % suspension of a fresh Mo promoted Raney-type Ni catalyst in a 50 wt % aqueous sorbitol solution, where the average particle size of the catalyst is ˜28 μm.

These tests were carried out with a fresh commercially available activated Ni catalyst that was doped with 1.2% Mo. This catalyst was used to prepare two 5wt % suspensions of this catalyst in 50 wt % aqueous sorbitol solutions. Each suspension was prepared by adding forty grams of this moist catalyst cake (23.5 grams on a dry basis) to 430 grams of a 50 wt % sorbitol solution. One suspension did not contain any additives and was used as a comparison, while the other was treated with 10 ml of a 0.05 wt % 806 BC cationic Praestol® solution. Both catalyst suspensions were stirred at room temperature to form homogeneous slurries and the stirring was stopped for both at the same time. The catalyst suspension with the 806 BC cationic Praestol® settled fast and produced a clear overstanding solution, however the catalyst slurry without the 806 BC cationic Praestol® settled slower and took more time to produce a clear overstanding solution. These two suspensions were then heated to 60° C., stirred to homogeneity and the stirring of both of them were stopped at the same time. At this elevated temperature the catalyst suspension with 806 BC cationic Praestol® settled faster and produced a clear overstanding solution much faster than the suspension without the flocculent.

### EXAMPLE 23

The use of flocculants for the improved settling properties of a 5 wt % suspension of a fresh Mo promoted Raney-type Ni catalyst in a 50 wt % aqueous glucose solution, where the average particle size of the catalyst is ˜28 μm.

These tests were carried out with a fresh commercially available activated Ni catalysts that was doped with 1.2% Mo. This catalyst was used to prepare three 5 wt % suspensions of this catalyst in 50 wt % aqueous glucose solutions. Each suspension was prepared by adding forty grams of this moist catalyst cake (23.5 grams on a dry basis) to 430 grams of a 50 wt % glucose solution. One suspension did not contain any additives and was used as a comparison, while the second one was treated with 5 ml of a 0.10 wt % 806 BC cationic Praestol® solution and the third one was treated with 5 ml of a 0.10 wt % 2515 anionic Praestol® solution. All three catalyst suspensions were stirred at room temperature to form homogeneous slurries and the stirring was stopped for all of them at the same time. The catalyst suspension with the 806 BC cationic Praestol® settled fast and produced a clear overstanding solution within 15 minutes. The catalyst suspension with the 2515 anionic Praesto® also settled fast and produced a clear overstanding solution within 15 minutes. However, the catalyst slurry without any flocculants settled slower and took longer to produce a clear overstanding solution. These suspensions were then heated to 60° C., stirred to homogeneity and the stirring of all three of them were stopped at the same time. At this elevated temperature the catalyst suspension with 806 BC cationic Praestol® settled fast and produced a clear overstanding solution in 15 minutes. The catalyst suspension with the 2515 anionic Praestol® also settled fast at 60° C. and produced a clear overstanding solution within 15 minutes. However like the room temperature experiment, the catalyst slurry at 60° C. without any flocculants settled slower and took longer to produce a clear overstanding solution in comparison to the suspensions with the flocculants.

### EXAMPLE 24

The use of flocculants for the improved settling properties of a fresh Mo promoted Raney-type Ni catalyst, where the average particle size of the catalyst is ˜53 μm.

100 grams of a Mo-doped Raney-type Ni catalyst (58.75 grams catalyst on a dry basis) were stirred in enough water to bring the total volume of this suspension up to 200 ml. Seven and a half ml of a 0.05 wt % Praestol® 2515 solution was then added to the catalyst suspension and it was stirred for 30 minutes. Forty grams of the resulting moist catalyst cake (23.5 grams on a dry basis) were weighed out and placed into a graduate cylinder. The graduate cylinder was filled to a volume of 100 ml with distilled water, a stopper was placed into the top of the graduate cylinder, it was shaken vigorously for 1 minute and the settling properties of the catalyst were then noted and measured. The catalyst was an agglomerating type that settled fast and the final level in the graduate cylinder was 35 ml giving a settled density of 1.14 g/ml for the catalyst cake. After 15 minutes of settling, the overstanding solution above the catalyst was clear. This catalyst is referred to as sample 24 in this patent.

### EXAMPLE 25

The use of flocculants for the improved embedding of a Raney-type Ni catalyst in distearylamine.

Eight hundred and fifty grams of a Raney-type Ni catalyst (500 grams catalyst on a dry basis) were stirred as part of an 800 ml slurry to form a homogeneous suspension. An aliquot of 212.5 ml of a 0.05 wt % Praestol® 852 BC was then added to the catalyst suspension and it was stirred for 10 minutes. Before the flocculant treatment, the moist settled catalyst cake had a density of 1.05 g/ml and after this treatment the density of the moist settled catalyst cake increased to 1.65 g/ml. In spite of the density increase, the flocculent treated catalyst exhibited agglomeration behavior during its settling which allowed it to settle faster than the untreated catalyst. This treated catalyst was initially allowed to settle and the overstanding solution was then removed via suction. The remaining moist catalyst was heated under vacuum to remove as much as possible of the remaining moisture followed by the addition of molten distearylamine, the homogenization of the mixture and the pastilation of this homogeneous mixture onto a cool surface to form embedded droplets of the modified catalyst in the secondary amine. The final concentration of the catalyst in the embedded mass was 60 wt %. In comparison to the original catalyst, the flocculant treated catalyst of this invention was much faster to embed as it settled faster and when it settled, the size of the catalyst bed was much smaller, meaning that the amount of water that could be siphoned off was much greater and the amount of water that had to be vacuumed off was much lower. Since siphoning is much faster and less energy intensive than evacuation, the flocculent treated catalyst is not only faster but it is also cheaper to embed. Because the flocculent treated catalyst also formed agglomerates, the homogenization process was also better meaning that the uniformity of the total catalyst embedding batch was improved.

### EXAMPLE 26

The use of flocculants for the improved embedding of a formaldehyde treated Raney-type Ni catalyst in monostearylamine.

Eight hundred and fifty grams of a Raney-type Ni catalyst (500 grams catalyst on a dry basis) were stirred as part of an 800 ml slurry to form a homogeneous suspension. Exactly 112,5 ml of an aqueous technical grade 37% formaldehyde solution were then added uniformly over 5 minutes to the catalyst slurry and this was followed by an additional hour of stirring. An aliquot of 250 ml of a 0.05 wt % Praestol® 852 BC was then added to the catalyst suspension and it was stirred for 10 minutes. Before the flocculant treatment, the formaldehyde modified catalyst settled very slow, it settled in a nonagglomerating fashion and the moist settled catalyst cake had a density of 1.90 g/ml. After flocculant treatment, the formaldehyde modified catalyst settled fast, it settled in an agglomerating fashion and the moist settled catalyst cake had a density of 1.81 g/ml. This formaldehyde modified and flocculant treated catalyst exhibited agglomeration behavior during its settling that allowed it to settle faster than the catalyst without flocculants. This treated catalyst was initially allowed to settle and the overstanding solution was then removed via suction. The remaining moist catalyst was heated under vacuum to remove as much as possible of the remaining moisture followed by the addition of molten monostearylamine, the homogenization of the mixture and the pastilation of this homogeneous mixture onto a cool surface to form embedded droplets of the modified catalyst in the primary amine. The final concentration of the catalyst in the embedded mass was 60 wt %. In comparison to the original catalyst, the flocculent treated catalyst of this invention was much faster to embed as it settled faster and when it settled, the size of the catalyst bed was essentially the same before and after this particular flocculant treatment, meaning that the amount of water that could be siphoned off and the amount of water that had to be vacuumed off was the same in both cases. Because the flocculant treated catalyst also formed agglomerates, the homogenization process was also better meaning that the uniformity of the total catalyst embedding batch was improved. In comparison to the catalyst that was not modified with formaldehyde, the catalyst of this invention generated far less ammonia and retained far more of the primary fatty amine during the embedding process and storage. Hence with the current invention, one is able to quickly embed a Raney-type Ni catalyst that has excellent settling properties that will also not convert the primary amine to secondary amines with the problematic evolution of ammonia. This methodology can also be used for the embedding of all types of formaldehyde modified Raney-type catalysts in both secondary and primary amines.

### EXAMPLE 27

The use of flocculants for the improved activation of a Raney-type Ni/Al alloy with NaOH.

Nine kilograms of an aqueous 20 wt % NaOH solution were mixed with 250 ml of a 0.05 wt % Praestol® 806 BC solution and heated to 95° C. 880 grams of a 53% Ni/47% Al alloy were then added to the heated mixture over a time period of one hour and this slurry was then stirred at this temperature for an additional 30 minutes. The freshly activated catalyst exhibited agglomeration behavior that led to a faster decantation of the activation solution and subsequent washing phase resulting in a catalyst with better settling behavior and a clearer overstanding solution when compared to normally activated catalysts.

### EXAMPLE 28

The use of flocculants for the improved washing of a freshly activated Raney-type Ni catalyst.

Nine kilograms of an aqueous 20 wt % NaOH solution were heated to 95° C. and 880 grams of a 53% Ni/47% Al alloy were then added to the heated mixture over a time period of one hour. This slurry was then stirred at this temperature for an additional 30 minutes. The freshly activated catalyst was then allowed to settle and the overstanding sodium aluminate/caustic solution was then siphoned off. In the meanwhile, one liter of water was mixed with 250 ml of a 0.05 wt % Praestol® 806 BC solution and this solution was then added to the freshly activated and decanted catalyst. The catalyst was then stirred in the diluted flocculant solution for 30 minutes followed by a settling period. Since the catalyst now exhibited agglomeration behavior, it settled very fast and this led to a faster washing phase and a clearer overstanding solution when compared to normally activated catalysts.

### EXAMPLE 29

The use of flocculants for the improved activation of a Raney-type Ni/Al alloy and the washing of the resulting freshly activated Raney-type Ni catalyst.

Nine kilograms of an aqueous 20 wt % NaOH solution were heated to 95° C. and 880 grams of a 53% Ni/47% Al alloy were then added to the heated mixture over a time period of one hour. This slurry was then stirred at this temperature for an additional 20 minutes, after which 250 ml of a 0.05 wt % Praestol® 806 BC solution was added and the slurry was stirred for a further 10 minutes as the suspension was allowed to cool. The freshly activated catalyst was then allowed to settle and the overstanding sodium aluminate/caustic solution was then siphoned off. Since the catalyst now exhibited agglomeration behavior, it settled very fast and this led to a faster decantation of the activation solution, catalyst washing phase and a clearer overstanding solution when compared to normally activated catalysts.

### EXAMPLE 30

The use of flocculants for the improved pumping of a Raney-type Ni/Al alloy water suspension.

It is advantageous sometimes to add the alloy to the activation solution as a water suspension and in this case, it is important that this suspension has well defined settling properties and is readily pumped. 880 grams of a 53% Ni/47% Al alloy were added over 10 minutes to a water solution that was prepared by the addition of 250 ml of a 0.05 wt % Praestol® 806 BC solution to 750 ml of water. The resulting suspension was easily reslurried after settling and it was readily pumped from the alloy suspension tank into the activation vessel without problems such as alloy compaction in the pump and suspension inhomogeneity regardless of the pumping speed. This alloy suspension was pumped over the time of one hour into the activation reactor containing 9 kilograms of an aqueous 20 wt % NaOH solution that was heated to 95° C. This slurry was then stirred at this temperature for an additional 30 minutes. The freshly activated catalyst was then allowed to settle and the overstanding sodium aluminate/caustic solution was then siphoned off. One liter of water was then added and stirred with the catalyst over 10 minutes after which it was allowed to settle so that this washing solution can be siphoned off. This washing step was repeated 2 more times. Since the catalyst now exhibited agglomeration behavior, it settled very fast and this led to a faster washing phase and a clearer overstanding solution when compared to normally activated catalysts.

### EXAMPLE 31

The use of flocculants for the improved Mo doping of an activated Raney-type Ni catalyst with MoO₃, where the average particle size is ˜53 μm.

Eight hundred and fifty grams of a moist Raney-type Ni catalyst (500 grams catalyst on a dry basis) were mixed with 220 ml of a 0.05 wt % Praestol® 806 BC flocculent solution and enough water to bring the total volume up to 800 ml. This mixture was then stirred for 30 minute, after which 13.5 grams of MoO₃ were then added to this slurry and it was stirred for an additional 30 minutes. It was found that the overstanding solution above the settled catalyst contain 0 ppm Mo. Forty grams of the moist catalyst cake (23.5 grams on a dry basis) were weighed out and placed into a graduate cylinder. The graduate cylinder was filled to a volume of 100 ml with distilled water, a stopper was placed into the top of the graduate cylinder, it was shaken vigorously for 1 minute and the settling properties of the catalyst were then noted and measured. The catalyst was an agglomerating type that settled fast and the final level in the graduate cylinder was 29 ml giving a settled density of 1.38 g/ml for the catalyst cake. After 15 minutes of settling, the overstanding solution above the catalyst was clear and it did not contain Mo. This catalyst is referred to as sample 31 in this patent.

### COMPARATIVE EXAMPLE 1

Raney-type Ni catalyst, that is post-activation doped with Mo.

A Raney-type Ni catalyst, that is post-activation doped with Mo (via an ammonium molybdate compound) to a level of 1.2 wt % and has an average particle size of 53 μm is used in comparison to those modified according to this invention. This catalyst is referred to as sample CE1 in this patent.

### APPLICATION EXAMPLE 1

The Slurry Phase Hydrogenation of Nitrobenzene.

The hydrogenation of nitrobenzene was carried out in a baffled glass reactor outfitted with a bubble inducing stirrer spinning at 2000 rpm over 1.5 grams (on a dry basis) of catalyst in 110 ml of a 9.1% nitrobenzene ethanolic solution at atmospheric pressure and 25° C. The results of these tests are shown in table 18.

TABLE 18
The results of nitrobenzene hydrogenation over the catalysts
of this patent as described by application example 1.

| Sample | Nitrobenzene hydrogenation activity |
|---|---|
| number | (ml H₂)/(min · gram catalyst) |
| 1a | 37 |
| 1b | 40 |
| 1c | 39 |
| 1d | 40 |
| 1e | 37 |
| 6a | 17 |
| 6b | 18 |
| 6c | 18 |
| 6d | 17 |
| 7a | 58 |
| 7b | 68 |
| 8a | 69 |
| 8b | 74 |
| 9a | 68 |
| 9f | 68 |
| 11a | 13 |
| 11b | 13 |
| 11c | 13 |
| 11d | 13 |
| 11e | 13 |
| 12a | 23 |
| 12b | 24 |
| 13b | 15 |
| 13c | 16 |
| 14 | 13 |
| 15 | 14 |

### APPLICATION EXAMPLE 2

The hydrogenation of butyronitrile.

The hydrogenation of butyronitrile was carried out in a baffled glass reactor outfitted with a bubble inducing stirrer spinning at 2000 rpm over 6 grams (on a dry basis) of catalyst in a solution containing 20 ml distilled water, 0.5 ml 50 wt % NaOH, 100 ml methanol and 10 ml butyronitrile at atmospheric pressure and 50° C. The results of these tests are shown in table 19.

TABLE 19
The results of butyronitrile hydrogenation over the catalysts
of this patent as described by application example 2.

| Sample | ml of hydrogen taken up after 60 minutes |
|---|---|
| number | of butyronitrile hydrogenation |
| 5a | 455 |
| 5b | 428 |
| 5c | 423 |
| 5d | 435 |

### APPLICATION EXAMPLE 3

The Slurry Phase Hydrogenation of Fructose.

Fructose was hydrogenated as 500 grams of its 40% aqueous solution at 50 bar in a 1 liter autoclave. The reaction temperature of 100° C. and 2.4 wt. % catalyst was used for the Raney-type Ni catalysts used here. The autoclave was initially charged with the catalyst and fructose solution followed by three purges with nitrogen and 4 purges with 5 bars of hydrogen. The reactor was then pressurized to 45 bars and agitation was started at 1015 rpm as the reaction mixture was heated from room temperature to the desired final reaction temperature. As reaction mixture was heated pressure built up due to the increased water vapor and once this pressure dropped due to the initial hydrogen consumption, the hydrogen pressure was then adjusted to 50 bars for the duration of the reaction. Samples were taken as the reaction progressed and these were analyzed via HPLC. The results of these tests are listed in table 20.

TABLE 20
The results from the slurry phase hydrogenation
of fructose over the catalysts of this patent as described
by application example 3.

| | Max | Starting | | | |
|---|---|---|---|---|---|
| Sample | activity | Temp., | Mannitol % | Reaction | Final |
| number | ml H₂/min | ° C. | Selectivity | Time, min | % Conversion |
| CE1 | 800 | 52 | 44 | 59 | 98.7 |
| 24 | 800 | 52 | 54 | 62 | 99.0 |

### APPLICATION EXAMPLE 4

The Slurry Phase Hydrogenation of Adiponitrile.

The slurry phase hydrogenation of adiponitrile was carried out in a one liter steel autoclave with 3 grams of catalyst (on a dry basis), 86.4 grams of adiponitrile, 314 grams of ethanol, 2 ml of a 30 wt % NaOH solution and 20 grams of water. After purging the reactor three times with nitrogen and three times with hydrogen, the autoclave was pressurized to 25 bar and stirred at 2000 rpm before starting the temperature ramp from room temperature to 75° C. over about 60 minutes. Once the reaction started, the reaction pressure was kept constant at 25 bar. Samples were taken from the reaction mixture for GC analysis at the reaction times of 0, 15, 30 and 45 minutes. After the reaction was stopped, the reaction mixture was separated from the catalyst and analyzed by GC. The results are listed in table 21. After the reaction, all of the flocculent treated catalysts exhibited improved settling behavior. It is also interesting to note that the flocculent treated catalysts performed superior to the catalyst that were not treated with flocculants. It is known that the addition of NaOH and other bases enhances the selectivity and the activity of these catalysts for the hydrogenation of adiponitrile to hexamethylenediamine due to the avoidance of strongly adsorbing Schiff bases that act as reversible poisons (please see for further details: D. J. Ostgard, M. Berweiler, S. Röder and P. Panster, in “Catalysts of Organic Reactions”, D. G. Morrell editor, Marcel Dekker, Inc., New York, 2002, 273-294). Surprisingly, the presence of the flocculant enhances the effectiveness of this reaction system even further. Hence, this technology could also be used to improve the interaction of the reactant to the catalyst surface for fixed bed catalysts as well.

TABLE 21
The results from the slurry phase hydrogenation
of adiponitrile over the catalysts of this patent as
described by application example 4.

| | Acti- | | | | | | |
|---|---|---|---|---|---|---|---|
| | vation | Reaction | | | | | |
| Sample | Energy | time | Percentage of reaction mixture* | | | | |
| number | KJ/mole | minutes | HMI | HMDA | ACN | ADN | Others |
| 7a | 32.8 | 0 | 0 | 0 | 0 | 100 | 0 |
| | | 15 | 0 | 18.86 | 56.09 | 25.05 | 0 |
| | | 30 | 0 | 100 | 0 | 0 | 0 |
| | | 45 | 0.54 | 99.46 | 0 | 0 | 0 |
| 7b | 33.4 | 0 | 0 | 0 | 0 | 100 | 0 |
| | | 15 | 0 | 19.48 | 55.86 | 24.66 | 0 |
| | | 30 | 0 | 96.75 | 3.25 | 0 | 0 |
| | | 45 | 0.42 | 99.58 | 0 | 0 | 0 |
| 8a | 33.8 | 0 | 0 | 0 | 0 | 100 | 0 |
| | | 15 | 0 | 24.56 | 57.43 | 18.01 | 0 |
| | | 30 | 0.43 | 99.57 | 0 | 0 | 0 |
| | | 45 | 0.55 | 99.45 | 0 | 0 | 0 |
| 8b | 33.4 | 0 | 0 | 0 | 0 | 99.99 | 0 |
| | | 15 | 0 | 25.65 | 56.62 | 17.73 | 0 |
| | | 30 | 0 | 100 | 0 | 0 | 0 |
| | | 45 | 0.43 | 99.56 | 0 | 0 | 0 |
| 9a | 37.5 | 0 | 0 | 0 | 0 | 100 | 0 |
| | | 15 | 0 | 9.61 | 50.76 | 39.63 | 0 |
| | | 30 | 0 | 100 | 0 | 0 | 0 |
| | | 45 | 0.56 | 99.44 | 0 | 0 | 0 |
| 9e | 39.4 | 0 | 0 | 0 | 0 | 99.99 | 0 |
| | | 15 | 0 | 25.81 | 61.86 | 12.33 | 0 |
| | | 30 | 0 | 100 | 0 | 0 | 0 |
| | | 45 | 0.49 | 99.51 | 0 | 0 | 0 |

*HMI = cyclohexamethyleneimine, HMDA = hexamethylenediamine, ACN = aminocapronitrile and ADN = adiponitrile.

### EXAMPLE 32

The production of a Cr and Fe promoted Raney-type Ni hollow spheres fixed bed catalyst and its treatment with flocculants

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the Cr and Fe promoted Ni/Al alloy and Ni binder onto a fluidized bed of styrofoam balls. This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5). 100 ml of the activated Cr and Fe doped Ni hollow spheres were placed in a basket immersed in 400 ml of a stirred water solution. 50 ml of a 0.05 wt % Praestol® 806 BC flocculent solution was then added to the solution and it was stirred for another hour and the resulting activated hollow spheres were then stored in a part of the resulting treatment solution. This catalyst is referred to as sample 32 in this patent.

### EXAMPLE 33

The production of a Cr and Fe promoted Raney-type Ni hollow spheres fixed bed catalyst.

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the Cr and Fe promoted Ni/Al alloy and Ni binder onto a fluidized bed of styrofoam balls. This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5). This catalyst is referred to as sample 33 in this patent.

### APPLICATION EXAMPLE 5

The trickle phase hydrogenation of adiponitrile over fixed bed catalysts.

Forty ml of the water protected activated hollow spheres where placed in a tube reactor that was initially purged with nitrogen followed by purging with hydrogen before the catalyst was dried under hydrogen. This hydrogenation was carried out in the trickle phase with a 20 wt. % adiponitrile in methanol solution at 65 bar, 113° C., and at the LHSV values of 0.26 and 1.03 h-1. The reactant also contained 1.9 grams of NaOH per liter of reaction feed. Table 22 shows the results of these tests. These results confirm those of application example 4 that were performed in the slurry phase.

TABLE 22
The results from the trickle phase hydrogenation
of adiponitrile over fixed bed Raney-type Ni catalysts as
described by application example 5.

| Sample | LHSV | | % Selectivity | | | |
|---|---|---|---|---|---|---|
| number | h⁻¹ | % Conversion | HMDA | ACN | 2° Amines | Rest |
| 33 | 1.03 | 79.1 | 70.6 | 20.7 | 6.3 | 2.4 |
| | 0.26 | 97.9 | 89.0 | 3.5 | 5.7 | 1.8 |
| 32 | 1.03 | 82.2 | 75.2 | 23.4 | 1.1 | 0.3 |
| | 0.20 | 99.8 | 94.4 | 4.0 | 1.3 | 0.3 |

LHSV is the liquid hourly space velocity (h⁻¹)
HMDA = hexamethylenediamine
ACN = aminocapronitrile

### APPLICATION EXAMPLE 6

The trickle phase hydrogenation of adiponitrile over fixed bed catalysts with flocculants in the feed.

Forty ml of the water protected activated hollow spheres where placed in a tube reactor that was initially purged with nitrogen followed by purging with hydrogen before the catalyst was dried under hydrogen. This hydrogenation was carried out in the trickle phase with a 20 wt. % adiponitrile in methanol solution at 65 bar, 113° C., and at the LHSV values of 0.26 and 1.03 h-1. The reactant also contained 1.9 grams of NaOH and 2 ml of a 0.05 wt % Praestol® 806 BC flocculant solution per liter of reaction feed. Table 23 shows the results of these tests.

TABLE 23
The results from the trickle phase hydrogenation
of adiponitrile over fixed bed Raney-type Ni catalysts as
described by application example 6.

| Sample | LHSV | | % Selectivity | | | |
|---|---|---|---|---|---|---|
| number | h⁻¹ | % Conversion | HMDA | ACN | 2° Amines | Rest |
| 33 | 1.03 | 80 | 72.1 | 21.5 | 4.8 | 1.6 |
| | 0.26 | 99 | 90.6 | 3.9 | 4.8 | 0.7 |

### EXAMPLE 34

The use of flocculants for the promotion of a Raney-type Ni hollow spheres fixed bed catalyst with Mo.

Activated Raney-type Ni hollow spheres were produced according to the patent literature (Ostgard et al U.S. Pat. No. 6,747,180, Ostgard et al U.S. Pat. No. 6,649,799, Ostgard et al U.S. Pat. No. 6,573,213 and Ostgard et al U.S. Pat. No. 6,486,366) by spraying an aqueous polyvinyl alcohol containing suspension of the Ni/Al alloy and Ni binder onto a fluidized bed of styrofoam balls. This spraying was performed in 2 steps. After impregnation, the coated styrofoam spheres were first dried and then calcined at 750° C. to burn out the styrofoam and stabilize the metal shell. The hollow spheres of alloy were then activated in a 20 to 30% caustic solution from 1.5 to 2 hours at ˜80 to 100° C. The catalyst was then washed and stored in a mildly caustic aqueous solution (pH ˜10.5). 100 ml of the activated Ni hollow spheres were placed in a basket immersed in 400 ml of a stirred water solution. 50 ml of a 0.05 wt % Praestol® 806 BC flocculant solution and 2.7 grams of MoO3 were then added to the solution and it was stirred for an hour. After stirring it was found that the treatment solution contained 0 ppm Mo, meaning that all of the Mo was successfully adsorbed on the catalyst's surface. This catalyst is referred to as sample 34 in this patent.

1. A process for the adjustment of a catalyst's or a catalyst precursor's suspension and settling properties, including:
settling rate after stirring of the suspension,
degree of agglomerate formation during settling of the catalyst,
settling density of the settled catalysts,
amount of and type of void volume inside the particle agglomerates,
size of the particle agglomerates,
appearance and content of the overstanding solution above the settled catalysts and
speed in which the settled catalyst can be resuspended,
comprising treating the catalyst with flocculants.

2. A process for the adjustment of a catalyst precursor's suspension and settling properties according to claim 1 for the optimal production of catalysts.

3. A process for the adjustment of a Raney-type alloy's suspension and settling properties by the addition of flocculants according to claim 1 for the optimal introduction of its water suspension into an activation reactor.

4. A process for the adjustment of the suspension and settling properties of a Raney-type catalyst activation suspension by the addition of flocculants according to claim 1 at the beginning, the end and/or during of the activation of the catalyst.

5. A process for the adjustment of the suspension and settling properties of a Raney-type catalyst wash suspension by the addition of flocculants according to claim 1 at the beginning, the end and/or during washing of the catalyst.

6. A process for the adjustment of the suspension and settling properties of a Raney-type catalyst by the addition of flocculants according to claim 1 at the end of a last wash step.

7. A process for the adjustment of the suspension and settling properties of a Raney-type catalyst by the addition of flocculants according to claim 1 to a drum of said catalyst.

8. A process for the adjustment of the suspension and settling properties of a Raney-type catalyst by the addition of flocculants according to claim 1 at the beginning, the end and/or during a catalytic reaction with said catalyst.

9. A process for the adjustment of the suspension and settling properties of a Raney-type catalyst by the addition of flocculants according to claim 1 at the beginning, the end and/or during the washing step of the catalyst after a catalytic reaction.

10. A process for the adjustment of the suspension and settling properties of a Raney-type catalyst by the addition of flocculants according to claim 1 at the beginning, the end and/or during the washing step of the catalyst after a catalytic reaction and before reuse.

11. A process for the adjustment of the suspension and settling properties of a catalyst that has been modified via the deposition of carbon containing residues with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in a liquid phase in the presence of one or more solvents as a treatment solution at temperatures ranging from above 0 to 150° C. for a period of time ranging from instant contact to longer than 24 hours when the catalyst is to be stored in the treatment solution by the addition of flocculants according to claim 1 at the beginning, the end and/or during carbon deposition step.

12. A process for the adjustment of the suspension and settling properties of a catalyst that has been modified via the deposition of carbon containing residues in the presence of one or more templates with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in a liquid phase in the presence of one or more solvents as a treatment solution at temperatures ranging from above 0 to 150° C. for a period of time ranging from instant contact to longer than 24 hours when the catalyst is to be stored in the treatment solution by the addition of flocculants according to claim 1 at the beginning, the end and/or during carbon deposition step.

13. A process for the adjustment of the suspension and settling properties of a Raney-type catalyst that has been modified via the deposition of carbon containing residues in the presence of one or more bases with a material selected from the group consisting of formaldehyde, metal salt formates, carbon monoxide, carbon dioxide, aldehydes, ketones, amides, carboxylic acids, salts of carboxylic acids and organic molecules that interact strongly with metal surfaces via their contact with the catalyst in a liquid phase in the presence of one or more solvents as a treatment solution at temperatures ranging from above 0 to 150° C. for a period of time ranging from instant contact to longer than 24 hours when the catalyst is to be stored in the treatment solution by the addition of flocculants according to claim 1 at the beginning, the end and/or during carbon deposition step.

14. A process for the adjustment of the suspension and settling properties of a Raney-type catalyst that has been modified via the deposition of carbon containing residues with material selected from the groupt consisting of formaldehyde and sodium formate in a liquid phase in the presence of one or more solvents as a treatment solution at temperatures ranging from above 0 to 150° C. for a period of time ranging from instant contact to longer than 24 hours when the catalyst is to be stored in the treatment solution by the addition of flocculants according to claim 1 at the beginning, the end and/or during carbon deposition step

15. A process for the adjustment of the suspension and settling properties of a Raney-type catalyst that has been modified via the deposition of carbon containing residues in the presence of one or more bases with a material selected from the group consisting of formaldehyde and sodium formate in a liquid phase in the presence of one or more solvents as a treatment solution and bases at temperatures ranging from above 0 to 150° C. for a period of time ranging from instant contact to longer than 24 hours when the catalyst is to be stored in the treatment solution by the addition of flocculants according to claim 1 at the beginning, the end and/or during carbon deposition step.

16. A process for the adjustment of a catalyst's or a catalyst precursor's suspension and settling properties according to claim 1, where the flocculent is added in the presence of a coadsorbent.

17. A process for the adjustment of a catalyst's or a catalyst precursor's suspension and settling properties according to claim 1, where the flocculent is added in the presence of a chiral template.

18. A process for the adjustment of a catalyst's or a catalyst precursor's suspension and settling properties according to claim 1, where a polyacrylamide is used as the flocculant.

19. A process for the adjustment of a catalyst's or a catalyst precursor's suspension and settling properties according to claim 1, where a cationic anionic or neutral polyacrylamide is used as the flocculant.

20. (canceled)

21. (canceled)

22. A process for the adjustment of a catalyst's or a catalyst precursor's suspension and settling properties according to claim 1, where a polyacrylic acid and/or derivatives thereof is/are used as the flocculent.

23. A process for the adjustment of a catalyst's suspension and settling properties according to claim 1, where a derivative of a polyacrylamide or polyacrylic acid containing one or more chiral centers is used as the flocculant.

24. A process for the adjustment of a catalyst's or a catalyst precursor's suspension and settling properties according to claim 1, where the flocculant is added to the catalyst slurry as a powder, or as a previously dissolved solution or as an emulsion.

25. (canceled)

26. (canceled)

27. A process for the adjustment of a catalyst's or a catalyst precursor's suspension and settling properties according to claim 1, where the flocculent is added as a part of a pretreated suspension of the catalyst, the catalyst precursor or a different material.

28. A process for the adjustment of a catalyst's or catalyst precursor's, suspension and settling properties according to claim 1, where the catalyst is a metal powder catalyst, a catalytic metal black, a metal borohydride, a Raney-type catalyst, a Ushibara-type catalyst and other nonsupported metal catalysts.

29. (canceled)

30. A process for the adjustment of a supported catalyst's or catalyst precursor's, suspension and settling properties according to claim 1, where the catalyst's or catalyst precursor's, settling properties are dependent on the charge-to-particle size ratio of the catalyst.

31. (canceled)

32. A process for the adjustment of a Raney-type catalyst's or Raney-type alloy's suspension and settling properties according to claim 1.

33. (canceled)

34. A process for the adjustment of a catalyst's or a catalyst precursor's suspension and settling properties according to claim 1, where the catalyst or catalyst precursor consists of one or more of the elements from the periodic groups 1 A, 2A, IIIB, IVB, VB, VIB, VIIB, VIII, IB, IIB, IIIA, IVA, VA and VIA.

35. A process for the adjustment of a catalyst's or a catalyst precursor's suspension and settling properties according to claim 1, where the catalyst or catalyst precursor consists of one or more of the elements from the periodic groups 1A, 2A, IIIB, IVB, VB, VIB, VIIB, VIII, IB, IIB, IIIA, IVA, VA and VIA and is doped with one or more elements from the periodic groups 1A, 2A, IIIB, IVB, VB, VIB, VIIB, VIII, IB, IIB, IIIA, IVA, VA and VIA.

36. A process for the adjustment of a catalyst's or a catalyst precursor's suspension and settling properties according to claim 1, where the catalyst or catalyst precursor consists of one or more of the elements from the periodic groups IVB, VB, VIB, VIIB, VIII and IB and is doped with one or more elements from the periodic groups 1A, 2A, IIIB, IVB, VB, VIB, VIIB, VIII, IB, IIB, IIIA, IVA, VA and VIA.

37. A process for the adjustment of a Raney-type catalyst's suspension and settling properties according to claim 1, where the Raney-type catalyst consists of one or more of the elements from the periodic groups VIII and IB and is doped with one or more elements from the periodic groups 1A, 2A, IIIB, IVB, VB, VIB, VIIB, VIII, IB, IIB, IIIA, IVA, VA and VIA.

38. A process for the adjustment of a Raney-type catalyst's suspension and settling properties according to claim 1, where the Raney-type catalyst consists of one or more of the elements from the periodic groups VIII and IB and is doped with one or more elements from the periodic groups VIB, VIIB, VIII and IB.

39. A process for the adjustment of a Raney-type catalyst's suspension and settling properties according to claim 1, where the Raney-type catalyst consists of one or more of the elements from the periodic groups VIII and IB.

40. A process for the adjustment of a Raney-type alloy's suspension and settling properties according to claim 1, where the Raney-type alloy consists of Al alloyed together with of one or more of the elements from the periodic groups VIII and IB and is doped with one or more elements from the periodic groups 1A, 2A, IIIB, IVB, VB, VIB, VIIB, VIII, IB, IIB, IIIA, IVA, VA and VIA.

41. A process for the adjustment of a Raney-type alloy's suspension and settling properties according to claim 1, where the Raney-type alloy consists of Al alloyed together with of one or more of the elements from the periodic groups VIII and IB and is doped with one or more elements from the periodic groups VIB, VIIB, VIII and IB.

42. A process for the adjustment of a Raney-type alloy's suspension and settling properties according to claim 1, where the Raney-type alloy consists of Al alloyed together with of one or more of the elements from the periodic groups VIII and IB.

43. The process according to claim 1 further comprising doping catalysts and catalyst precursors with one or more elements from the periodic groups 1 A, 2A, IIIB, IVB, VB, VIB, VIIB, VIII, IB, IIB, IIIA, IVA, VA and VIA.

44. The process according to claim 28 further comprising doping the catalysts and catalyst precursors with one or more elements from the periodic groups 1A, 2A, IIIB, IVB, VB, VIB, VIIB, VIII, IB, IIB, IIIA, IVA, VA and VIA.

45. (canceled)

46. The process according to claim 28 further comprising doping the catalysts with one or more elements from the periodic groups VIB, VIIB, VIII and IB.

47. The process according to claim 1 further comprising doping a Raney-type catalyst consisting of one or more of the elements from the periodic groups VIII and IB wherein said doping is with one or more elements from the periodic groups 1A, 2A, IIIB, IVB, VB, VIB, VIIB, VIII, IB, IIB, IIIA, IVA, VA and VIA.

48. The process according to claim 1 further comprising doping a fixed bed Raney-type catalyst consisting of one or more of the elements from the periodic groups VIII and IB wherein said doping is with one or more elements from the periodic groups 1A, 2A, IIIB, IVB, VB, VIB, VIIB, VIII, IB, IIB, IIIA, IVA, VA and VIA.

49. The process according to claim 1 further comprising doping a Raney-type catalyst consisting of one or more of the elements from the periodic groups VIII and IB wherein said doping is with one or more elements from the periodic groups VIB, VIIB, VIII and IB.

50. The process according to claim 1 wherein flocculants are applied to a fixed bed catalyst to improve the reactant-to-catalyst interface or to improve the reaction additive-to-catalyst interface.

51. (canceled)

52. The process according to claim 1 further comprising embedding the catalysts in fatty amines or polyethyleneglycol.

53. The process according to claim 1 further comprising embedding the catalysts in monostearyl amine, distearyl amine, tallow amine or polyethyleneglycol.

54. The catalysts produced by the process of claim 52.

55. A process for the transformation of organic compounds comprising using the catalysts according to claim 1.

56. A process for the hydrogenation of unsaturated compounds comprising using the catalysts according to claim 1.

57. A process for the improved enantioselective hydrogenation of prochiral unsaturated compounds comprising using the catalysts according to claim 1.

58. A process for the hydrogenation of nitro groups in organic compounds comprising using the catalysts according to claim 1.

59. A process for the hydrogenation of nitrile groups in organic compounds comprising using the catalysts according to claim 1.

60. A process for the hydrogenation of sugars to polyols comprising using the catalysts according to claim 1.

61. A process for the improved enantioselective hydrogenation of fructose to mannitol comprising using the catalysts according to claim 1.

62. A process for the hydrogenation of carbonyl groups in organic compounds comprising using the catalysts according to claim 1.

63. A process for the hydrogenation of dinitriles comprising using the catalysts according to claim 1.

64. A process for the hydrogenation of adiponitrile comprising using the catalysts according to claim 1.

65. A process for the use of flocculants comprising adding flocculants to production waste streams of a catalyst or to catalytic reaction waste streams for the faster and clearer sedimentation of catalyst fines.

66. (canceled)

67. A process for the use of flocculants for the more effective interaction in a system between a catalyst and a reactant or between a catalyst and a reaction's additive or between a catalyst, a reactant and the reactant's additive comprising adding a flocculant to said system.

68. (canceled)

69. (canceled)
